(19) United States
(12) Patent Application Publication
(10) Pub. No.: US 2007/0049996 A1
(43) Pub. Date: Mar. 1, 2007

(54) Monitoring Method and Apparatus for Fractional Photo-Therapy Treatment

(75) Inventor: John F. Black, San Mateo, CA (US)

Correspondence Address: FENWICK & WEST LLP, SILICON VALLEY CENTER, 801 CALIFORNIA STREET, MOUNTAIN VIEW, CA, 94041, US

(73) Assignee: RELIANT TECHNOLOGIES, INC., Mountain View, CA (US)

(21) Appl. No.: 11/468,264

(22) Filed: Aug. 29, 2006

Related U.S. Application Data
(60) Provisional application No. 60/712,660, filed on Aug. 29, 2005.

(51) Int. Cl. A61N 5/06

(52) U.S. Cl. 607/89

(57) ABSTRACT

A monitoring method for tissue treatment by fractional photo-therapy includes recording a fluorescence image of an area of tissue being treated and electronically processing the image to provide a measure of either progress of the treatment or an applied treatment radiation dose. Fluorescence is generated by irradiating the tissue with ultraviolet (UV) or blue radiation to stimulate fluorescence of one or more chromophores in the tissue. The monitoring method may be applied to control a treatment light source in phototherapy apparatus. In one example of phototherapy apparatus, a handpiece for delivering treatment light to the tissue includes a source of the UV radiation and a CCD camera for recording the fluorescence image.

### CROSS-REFERENCE TO RELATED APPLICATION

This application claims priority under 35 U.S.C. § 119(e) to U.S. Provisional Patent Application Ser. No. 60/712,660, “Monitoring Method And Apparatus For Fractional Photo-Therapy Treatment,” filed Aug. 29, 2005. The subject matter of all of the foregoing is incorporated herein by reference in their entirety.

### TECHNICAL FIELD OF THE INVENTION

The present invention relates in general to photo-thermal treatment of human skin. The invention relates in particular to a method for monitoring the progress of fractional photo-thermal treatment during or immediately following the treatment.

### DISCUSSION OF BACKGROUND ART

Fractional photo-thermal treatment (fractional photo-therapy) involves creating microscopic treatment zones (MTZs) of necrotic tissue with the MTZs being surrounded by annuli of viable tissue that may be thermally shocked. These annuli of viable tissue may be separated from each other by spared tissue. Treatment apparatus includes one or more light sources and a delivery system to generate the MTZs in a predetermined pattern. The MTZs may be confined to the epidermis, dermis or span the epidermal-dermal junction. Further, the stratum corneum above the microscopic treatment zones may be spared.

Several embodiments of method and apparatus for fractional photo-therapy are described in detail in published U.S. Patent Applications 20050049582 and 20030216719, the complete disclosures of which are hereby incorporated by reference. A brief description of certain aspects of the '582 application is set forth below to provide a contextual reference for the present invention, beginning with reference to FIG. 1 and FIG. 2.

FIG. 1 is a cross-sectional view schematically illustrating a fragment 10 of human skin being treated by the fractional photo-therapy method of the '582 application. The skin comprises the dermis 12 surmounted by the epidermis 14, with an irregular boundary 16 between the dermis and the epidermis. The epidermis is covered by the stratum corneum 18. At the base of the dermis is subcutaneous tissue 20. Microscopic laser beams 22 are directed into the skin and can penetrate into the dermis. The microscopic laser beams have sufficient power to coagulate tissue and kill cells in the path of the beams, creating zones 24 of necrotic tissue. The necrotic tissue zones or MTZs are separated by viable tissue 26. Depending on the wavelength, power, and focusing of radiation in laser beams 22, the MTZ may extend completely through the epidermis 14 into the dermis 12. In this case, it is possible to spare the stratum corneum by appropriate beam focusing and choice of radiation parameters. Surface cooling can be used to provide that necrotic tissue zones 24 occur only in the dermis 12.

FIG. 2 is a view seen generally in a plane 2-2 of FIG. 1, schematically illustrating the general form of a hypothetical, two-dimensional array of spaced-apart necrotic tissue zones or MTZs 24 formed in a fractional photo-therapy treatment. Each of the MTZs 24 is surrounded by tissue, with a zone 28 of the tissue being thermally shocked by the delivery of the laser beam but nevertheless still viable. In this thermally shocked zone, a wound-healing response occurs, causing the growth of new tissue. The necrotic tissue is eventually replaced with new tissue. Treatments for various skin conditions are possible depending on the wavelength of radiation and the location of the zones of necrotic tissue.

One embodiment of prior-art apparatus for effecting fractional photo-therapy treatment of skin 10 is schematically depicted in FIG. 3. Here, treatment apparatus 30 includes a diode-laser array radiation source 32 for providing treatment radiation. Such radiation source would include a plurality of individual diode-lasers, either in a one dimensional array (diode-laser bar), or a stack of such arrays. In apparatus 30 it is assumed that radiation is delivered as pulses of radiation. Radiation from source 32 is transported via an optical fiber 34 to a treatment handpiece 36. In handpiece 36, a coupler 38 spreads the diode-laser radiation into a beam 40 to be incident on an array 42 of microlenses 44. Each microlens 44 focuses a particular portion of the incident beam 40 to create the plurality of individual beams 22. Beams 22, in turn, create MTZs 24 in skin 10 being treated, as discussed above. In this particular embodiment of apparatus 30, handpiece 36 includes a skin-cooling plate 43 for sparing the stratum corneum 18 and other epidermal tissue from thermal destruction.

Microlens array 42 may be a one-dimensional or a two-dimensional array. In a handpiece with a one-dimensional microlens array, a two-dimensional array of MTZs can be produced by moving the handpiece in a direction perpendicular to length of the microlens array, while triggering a pulse of radiation in each new position of the microlens array.

In other embodiments, an optical scanning delivery system is used instead of or in addition to microlens array 42. An example of a scanning delivery system is a galvanometer scanner or a starburst scanner as described in copending application 60/652,891 “Optical pattern generator using a single rotating component” that is incorporated herein by reference.

A particular advantage of the fractional photo-therapy method, compared with prior-art skin therapy or rejuvenation treatments such as laser skin exfoliation or ablation, is that treatment can be effected without a patient requiring significant “down time” to require a skin wound to heal, or without the patient exhibiting unsightly scars or visible inflammation of the skin for a prolonged period of time after the treatment. In this regard, it is contemplated that the fractional photo-therapy treatment is applied two or more times, at selected time intervals between treatments, to an area of skin being treated, until a desired result has been attained. In order to achieve the desired result with a minimum of such repeat treatments, it would be useful to be able to monitor the effectiveness of any particular treatment. Such monitoring could be performed during treatment so as not to over- or under-treat a selected area of skin being treated. The monitoring could also be performed between treatments to gauge the optimum interval for subsequent treatments.

### SUMMARY OF THE INVENTION

The present invention is directed to a method and apparatus for monitoring progress of fractional photo-therapy treatment. In the fractional photo-therapy treatment, treatment radiation is delivered in a pattern of spaced-apart zones to an area of tissue. In one aspect, the method comprises irradiating the area of tissue with electro-magnetic radiation, thereby stimulating emission of fluorescence radiation from one or more fluorophores in the area of tissue. One or more images of the area of tissue are recorded using the fluorescence radiation emitted from at least one of the fluorophores. The images have a spatial distribution of fluorescence depending on structural features of the area of tissue combined with features characteristic of the pattern of treatment-radiation delivery. At least one of the one or more images is electronically processed to identify that portion of the fluorescence radiation in the one or more images resulting from the delivery of the treatment radiation.

The treatment radiation portion of the one or more images can be interpreted as a measure of the effectiveness or progress of the fractional-phototherapy treatment. Alternatively the treatment radiation portion of the one or more images can be interpreted as a measure of the dose of treatment radiation delivered to the area of tissue to be treated. In a fractional photo-therapy apparatus, one or more of these measures may be used to control a treatment-light source providing the treatment radiation.

In one preferred embodiment of the method, wherein treatment radiation is delivered in a pattern of regularly (periodically) spaced zones thereof, an image is recorded using fluorescence radiation in a band of wavelengths characteristic of the fluorophore reduced nicotinamide adenine dinucleotide (NAD-H). The image is electronically processed by generating a Fourier transform of at least one region of the image. The portion of the image fluorescence resulting from the delivery of treatment radiation is represented by a peak of the Fourier transform. The amplitude of the Fourier transform peak can be interpreted as a measure of the effectiveness of the fractional photo-therapy treatment, or as a measure of the treatment radiation dose delivered to the area of tissue.

### BRIEF DESCRIPTION OF THE DRAWINGS

The accompanying drawings, which are incorporated in and constitute a part of the specification, schematically illustrate a preferred embodiment of the present invention, and together with the general description given above and the detailed description of the preferred embodiment given below, serve to explain the principles of the present invention.

FIG. 1 is a cross-section view schematically illustrating principles of a prior-art fractional photo-therapy treatment for human skin, wherein a plurality of laser beams provides a plurality of necrotic tissue zones in the skin, the necrotic tissue zones having viable tissue therebetween.

FIG. 2 is a view seen generally in a plane 2-2 of FIG. 1, that schematically illustrates the general form of a hypothetical two dimensional array of spaced-apart necrotic tissue zones formed according to the principle of FIG. 1, with each of the necrotic tissue zones being surrounded by a heat-shock zone of thermally altered tissue in which a healing response occurs, the healing response being characterized by chemical, cellular, and morphology changes in the thermally altered tissue, for example producing local, spatially selective changes in the levels of reduced nicotinamide adenine dinucleotide (NAD-H).

FIG. 3 schematically illustrates one example of prior-art apparatus for carrying out fractional photo-therapy in accordance with the principle of FIG. 1, the apparatus including a handpiece arranged to receive a primary laser beam and form that beam into a plurality of secondary laser beams for creating the plurality of necrotic tissue zones.

FIG. 4 schematically illustrates one embodiment of fractional photo-therapy apparatus in accordance with the present invention having a handpiece including a source of ultraviolet (UV) or blue radiation, directing the UV/blue radiation onto skin being treated by a plurality of laser beams, and a CCD camera for recording, via fluorescence generated in response to the UV/blue irradiation, a pixelated image of the skin being treated.

FIG. 5 schematically illustrates a fragment of a hypothetical “ideal” fluorescence-image of skin treated by one particular pattern of fractional phototherapy in apparatus similar to the apparatus of FIG. 4.

FIG. 6 is a graph schematically illustrating hypothetical “ideal” response signals of a row of CCD pixels in the fluorescence image of FIG. 5, with the row of pixels being aligned with a row of spaced-apart necrotic tissue zones in the skin being treated, the response having a periodic structure corresponding to the spaced-apart necrotic tissue zones and thermally-altered tissue zones surrounding same.

FIG. 7 is a graph schematically illustrating an estimated practical response of the row of pixels of FIG. 6, wherein the ideal response is distorted by random fluorescence having a peak amplitude equal to the brightest signal amplitude of the graph of FIG. 6.

FIG. 8 is graph similar to the graph of FIG. 7, but wherein the amplitude of the brightest ideal signal is 50% greater than that in the graph of FIG. 7.

FIG. 9 is a graph schematically illustrating a frequency spectrum (Fourier Transform) of the graph of FIG. 7, with one peak-frequency having an amplitude corresponding to the brightness of fluorescence in the thermally-altered tissue zones.

FIG. 10 is a graph schematically illustrating a frequency spectrum (Fourier Transform) of the graph of FIG. 8, with one peak-frequency having an amplitude corresponding to the brightness of fluorescence in the thermally-altered tissue zones.

FIG. 11 schematically illustrates another embodiment of apparatus in accordance with the present invention.

FIG. 12 schematically illustrates yet another embodiment of apparatus in accordance with the present invention.

FIG. 13 schematically illustrates still another preferred apparatus in accordance with the present invention.

FIG. 14 schematically illustrates a preferred embodiment of fractional phototherapy treatment apparatus in accordance with the present invention controlled by monitoring fluorescence from skin being treated.

### DETAILED DESCRIPTION OF THE INVENTION

The present invention relies on detecting changes in molecular composition, cellular activity, or tissue morphology that is caused by fractional photo-thermal treatment or corresponds to the wound healing response, for example the inflammatory response, triggered by a fractional photo-thermal treatment. The changes associated with the wound healing response are manifested by the changes in intensity of certain fluorophores in the thermal shock zones surrounding the zones of necrotic tissue, or by the appearance of new fluorophores, or by the disappearance of intrinsic fluorophores, or by shifts in the excitation/emission spectra of the fluorophores, or by changes in the polarization properties of the fluorescence. This activity can be monitored by stimulating fluorescence of the fluorophores. This stimulation is provided by irradiating the skin being treated with UV/blue wavelengths in the electromagnetic radiation spectrum. The stimulated fluorescence is used to form an image of the skin being treated. The image includes characteristics resulting from the stimulating wavelength, the fluorescence spectrum of the fluorophores, and the spatial distribution of treatment radiation which is characteristic of the fractional phototherapy process. This fluorescence image is electronically processed to provide an estimate of the effectiveness of the treatment.

The physiological well-being level in tissue is known to be related to mitochondrial activity in cells of the tissue. An integral part of this mitochondrial activity is the production of the fluorophore NAD-H (reduced nicotinamide adenine dinucleotide). In the above-discussed MTZs (necrotic tissue zones) of fractional photo-therapy treatment, the concentration of NAD-H is likely to increase following treatment due to the reduced perfusion of oxygen to the region and the reduction in ATP (adenosine tri-phosphate) turnover. These two processes will shift the mitochondrial redox potential to the reduced form of NAD(H) which is the fluorescent form. The oxidized form (NAD(+)) has no intrinsic fluorescence.

For the cells in the thermal shock (thermally-altered) zones surrounding the MTZs or fractional photo-therapy treatment, mitochondrial activity can be expected to increase significantly, resulting in an increase in ATP turnover and increased O₂ perfusion, driving the redox equilibrium to the oxidized state of NAD(+) and in turn lowering the fluorescence intensity. This decrease in fluorescence can be expected to occur promptly after treatment, as part of the wound healing response, although it may be delayed by latency in the biochemical marker expression process.

While NAD-H is a particularly preferred fluorophore to be monitored in accordance with the present invention, there are other intrinsic fluorophores in skin tissue that may be affected by fractional phototherapy and that could be monitored either individually or to supplement monitoring of NAD-H. TABLE 1 lists a selection of such fluorophores (including NAD-H). Also listed in TABLE 1 is the probable relationship of the fluorophores to fractional phototherapy, how the fluorophores might be expected to behave in and around the MTZs, and what the optimum excitation/emission wavelength pairs are for each fluorophore.

It is noted in TABLE 1 that the fluorophore elastin is not expected to show much change upon fractional phototherapy, however this can be used to advantage in comparing areas and isolating changes. By comparing the ratio of the fluorescence intensity of two fluorophores, one of which is expected to change and one which is not expected to change with treatment, the sensitivity of the inventive monitoring technique may be increased by calibrating out variations in a fluorescence image that are not due to the fractional photo-therapy treatment.

It is important in the inventive monitoring method that the method and apparatus employed be able to distinguish increased mitochondrial activity resulting immediately from the fractional photo-therapy treatment, from any normal mitochondrial activity that could be detected in the skin prior to the treatment. An example of the manner in which this distinguishing can be achieved is included in a detailed description of the invention set forth below.

Continuing with reference to the drawings, wherein like components are designated by like reference numerals, FIG. 4 schematically illustrates one preferred embodiment 50 of a fractional photo-therapy handpiece for implementing the monitoring method of the present invention. Handpiece 50 includes a one-dimensional microlens array 42, here, seen perpendicular to the length of the array. Each microlens 44 of the array provides a beam 22 for providing an MTZ as discussed above. A two-dimensional array of MTZs is produced by moving handpiece 50 forward or backward in a direction perpendicular to the length of the microlens array as indicated in FIG. 4 by arrow A.

TABLE 1

| | Components of | Excitation | Emission | |
|---|---|---|---|---|
| | tissue that change | Wavelength | Wavelength | |
| Fluorophore | in response to FP | (nm) | (nm) | Expected change |
| Tryptophan | Amino acid, | 295 | 360 | Proteins are denatured in the |
| | constituent of | | | micro-thermal zones, which may |
| | protein | | | cause a shift in the excitation/ |
| | | | | emission (EE) spectrum. |
| Porphyrins | Pigments, Blood | 400 | 630, 660 | Coagulation and denaturation. Loss |
| | | | | of fluorescence or shift in EE |
| | | | | spectra. Chemical change of oxy- |
| | | | | hemoglobin to met-hemoglobin |
| | | | | giving a change in absorption |
| | | | | spectrum. |
| NADH | Mitochondrial | 340 | 460 | Increase in necrosed zones, |
| | activity | | | decrease in heat-shocked zones. |
| Flavins (for | Mitochondrial | 400 | 525 | Decrease in mitochondrial oxygen |
| example, Flavin | activity | | | tension through loss of perfusion |
| Adenine | | | | and cell death leads to increased |
| Dinucleotide/ | | | | fluorescence from the oxidized |
| Mononucleotide) | | | | form of FAD and FMN |
| Elastin | Structural (Cell and | 350 | 420 | Little or no change expected. |
| | Tissue morphology | | | |
| Collagen | Structural (Cell and | 340 | 400 | Chemical denaturation leading to |
| | Tissue | | | shift of EE spectrum. Loss of |
| | morphology) | | | birefringence leading to changes in |
| | | | | the polarization properties of |
| | | | | collagen fluorescence. |

Handpiece 50 includes a source 52 of ultraviolet/blue radiation, preferably having a wavelength between about 290 and 400 nanometers (nm) depending on the fluorophore to be probed. For example, when probing NADH, the excitation wavelength is preferably between 300 and 385 nm, and, more preferably, between about 340 nm and 360 nm. Wavelengths in these ranges can be provided, for example, by light-emitting diodes (LEDs) or laser diodes having one or more indium gallium nitride (InGaN) or gallium nitride (GaN) active layer. These wavelengths can also be provided by eximer lasers, mercury arc lamps, tripled Nd:YAG lasers, tripled tunable Ti:sapphire lasers, or free-electron lasers. Two sources may be combined when a ratiometric comparison between two fluorophores is desired. Ultraviolet radiation 54 from source 52 is incident on skin 10 being treated. Fluorescence radiation 56, resulting from the irradiation of skin 10 by ultraviolet radiation 54, is imaged by a CCD camera 58. In FIG. 4, and in similar drawings discussed further hereinbelow, the direction of fluorescence-stimulating radiation 54, and the direction of resulting fluorescence 56, is identified by single and double open arrowheads respectively. The direction of treatment radiation is indicated by single solid arrowheads.

Processing electronics 60 are connected to CCD camera 58. These electronics are used for processing fluorescence images to determine increased fluorescence resulting from the fractional photo-therapy treatment. The image-processing electronics are depicted here as being separate from the CCD camera for convenience of description, but could simply be included in the CCD camera as a functional element thereof. Imaging optics are also assumed to be included in CCD camera 58 as needed.

A bandpass filter 62 is provided for limiting the bandwidth of radiation received by the camera to that which is characteristic of the fluorophore being imaged. By way of example, a filter transmitting wavelengths between about 420 and 550 nm is preferred when the target fluorophore is NAD-H. A bandpass filter having a peak transmission centered at 460 nm, and having a full bandwidth at half maximum transmission (FWHM) of between about 15 nm and 40 nm, is particularly preferred for imaging NAD-H fluorescence. Other bandpass filters may be selected for other fluorophores as described in TABLE 1.

Analysis of the data may take several forms. By way of example, using multiple excitation sources and multiple detected wavelengths, data on the extent of the treatment may be extracted using formalisms developed for hyperspectral imaging, and in particular, the Mahalanobis distance. Preferably, spatial domain imaging may be used to interpret the image data using techniques developed for image analysis.

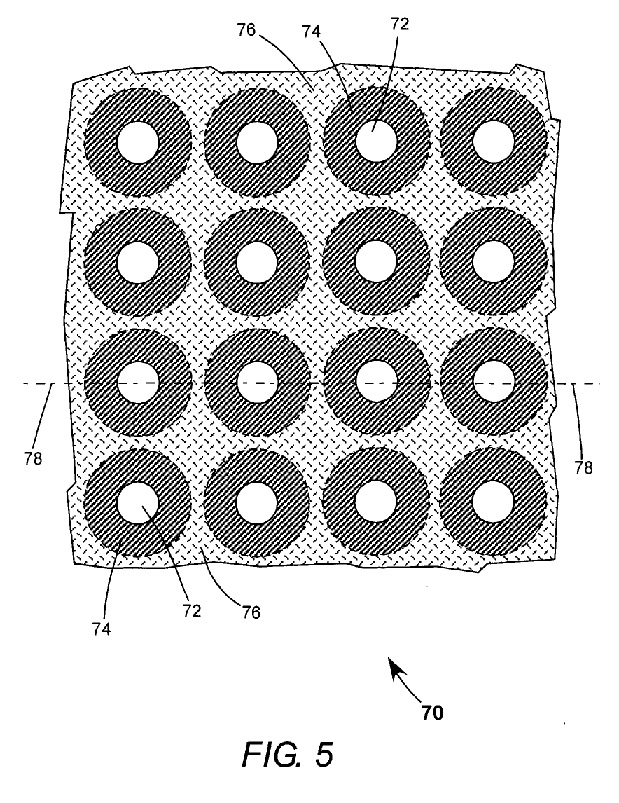

A description of one spatial analysis technique, usable in the method of the present invention to distinguish increased mitochondrial activity resulting immediately from the fractional photo-therapy treatment from any normal mitochondrial activity that could be detected in the skin prior to the treatment, is next presented beginning with reference to FIG. 5, which depicts a fragment 70 of a hypothetical (and essentially unobtainable) “ideal” fluorescence-image of the treated skin. The analysis is described, with reference to probing NAD-H in mitochondria, however those skilled in the art will recognize that the image analysis of the inventive monitoring method could also be applied to probing other fluorophores, including, but not limited to, fluorophores described in TABLE 1.

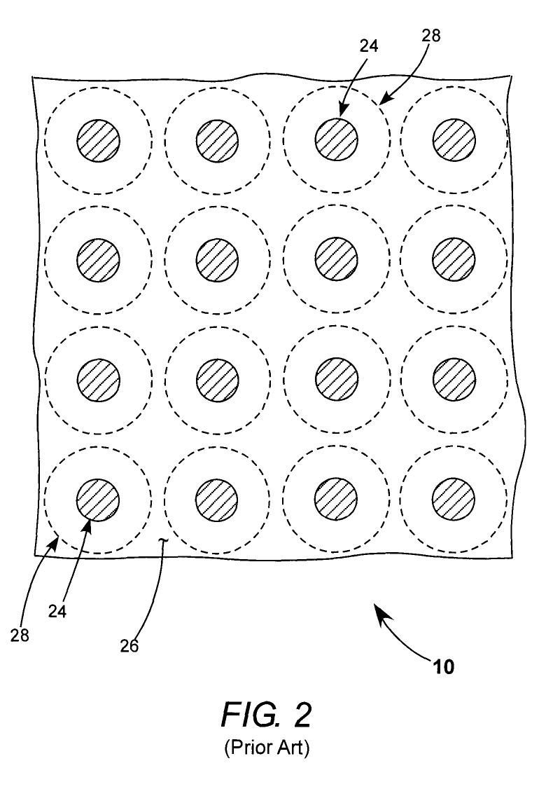

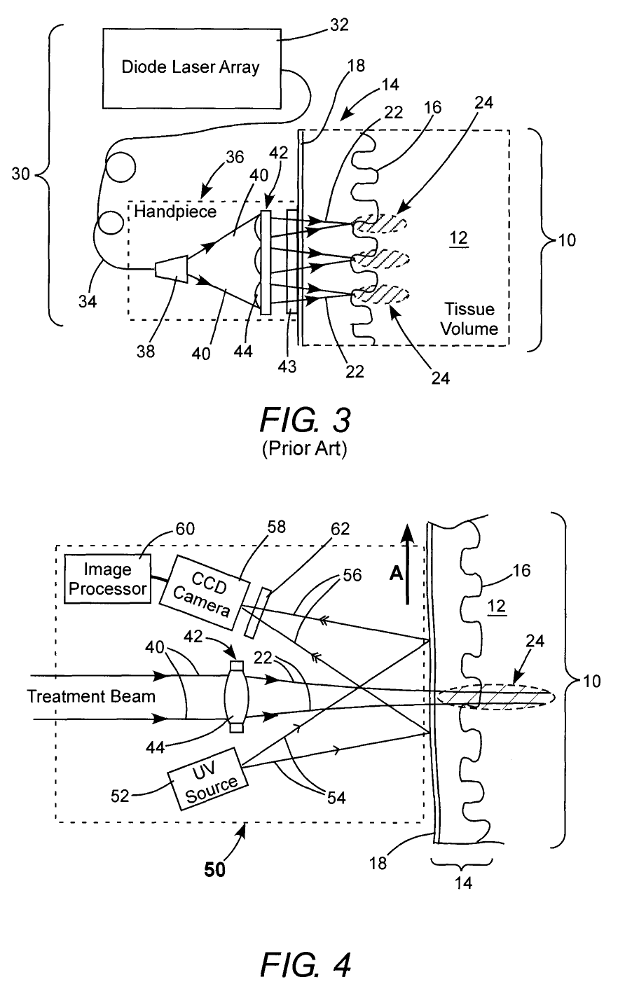

Continuing with reference to FIG. 5, hypothetical fluorescence-image 70 includes a plurality of bright zones 72 corresponding to MTZs 24 of FIG. 2; a plurality of annular, darker 74 corresponding to thermally shocked, but potentially viable zones 26 of FIG. 2, in which mitochondrial activity has been increased in response to the wound generated by the fractional photo-therapy treatment: and a less dark background area 76 where mitochondrial activity is “normal”, i.e., not significantly increased or decreased by the treatment. Here, it should be noted that, in fractional photo-therapy, treatments are contemplated in the above discussed '528 application in which thermally altered tissue zones overlap such that there would be no “normal” background.

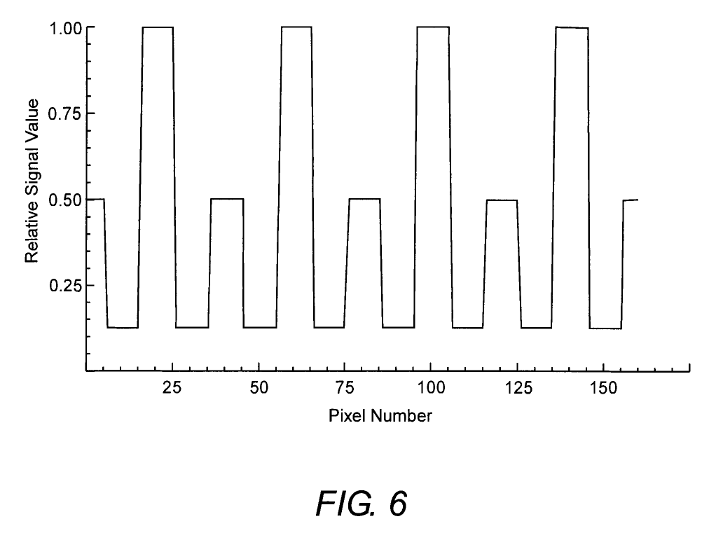

FIG. 6 is a graph schematically depicting a hypothetical signal level per pixel (of CCD camera in 160) of pixels aligned through the centers of the imaged bright zones 72, as indicated in FIG. 5 by dashed line 78. Pixels imaging bright zones 72 have been arbitrarily assigned a value of 1.0, with pixels imaging dark zones 74 and background zones 76 having arbitrarily-assigned values of 0.125 and 0.5 respectively. A reason for the selection of values in FIG. 6 is as follows.

In untreated tissue zones 76 surrounding each MTZ 72, normal metabolism creates a particular concentration balance between the reduced NADH and its oxidized state. In the stimulated regions 74 around the necrosed zone 72, the cell metabolism increases, which causes higher conversion of NADH from the reduced form to the oxidized state. The reduced form NADH is the only fluorescent state for the NADH, which means that the fluorescence in these regions is reduced relative to that in untreated tissue regions 76. It is also possible that the untreated zones between MTZs will experience some cell metabolism increase as a collateral effect (being proximate to a heat shocked zone), so that the fluorescence from NAD-H will also be reduced in the untreated areas.

In the regions 72, the opposite happens. In these regions, the tissue is coagulated so there is no longer a viable metabolic cycle converting reduced NADH to its oxidized state. Thus the reduced (fluorescent) form of NAD-H (is expected to?) will accumulate in higher concentration than in untreated tissue.

Those skilled in the art will recognize that the image of FIG. 5, and the corresponding pixel array graph of FIG. 6, in practice, would appear somewhat different than illustrated in FIG. 5 and FIG. 6. At a minimum, necrotic, thermally-altered, and background zones would probably not be of uniform brightness; and boundaries between zones would be blurred. Further there may be image structure present in addition to any periodicity of the image resulting from the array of MTZs. Factors influencing the practical image-appearance include scattering of the treatment, stimulating, and fluorescence radiations by the skin; variation of intensity in the treatment and stimulating radiation beams; the extent and depth of the MTZs in the skin; and the fact that fluorescence spectra of other fluorophores present in the skin can overlap the spectrum of the fluorophore being monitored. Variability of the skin itself must also be considered both on a microscopic scale, for example, around the pilo-sebaceous units, and a more macroscopic scale, for example, comparing skin on the face to that on the neck or hands. These factors, and other factors, would contribute to distorting or even obscuring (to the eye at least) any periodicity of the image that would be expected from the regular distribution of the MTZs.

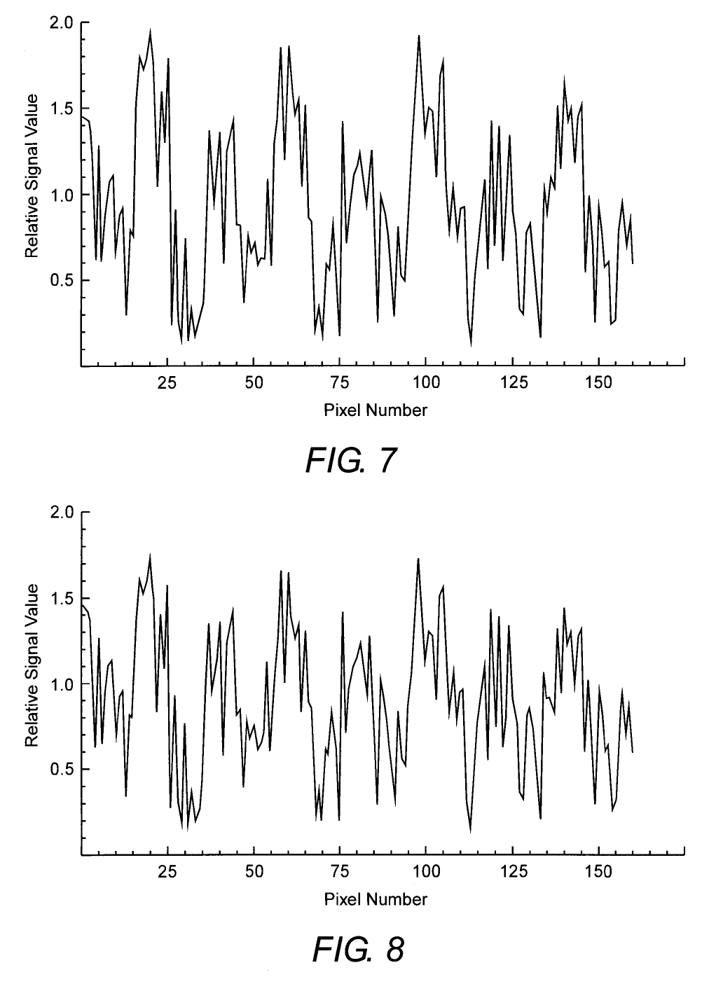

FIG. 7 is a graph schematically illustrating a mathematical simulation of significant image distortion that adds to the graph of FIG. 6 an artificial “untreated background” comprising a normally-distributed, random signal having a peak brightness equal to the brightness of the bright zones of FIG. 6. It can be seen that the added noise makes the thermally-altered zones and unaltered zones of FIG. 6 essentially indistinguishable throughout. FIG. 8 is a graph similar to the graph of FIG. 7 but wherein the “ideal” brightness of the bright zones and dark zones have been respectively decreased and increased by 20% (of the corresponding FIG. 6 values) to simulate an 20% less effective healing response than that of the graph of FIG. 6. Such a decrease could result, for example, from a decrease in energy or intensity of treatment radiation delivered to the MTZs. The random background of FIG. 8 is the same random background as that of FIG. 7.

One simple method of processing the “line” images represented by the graphs of FIG. 7 and FIG. 8 to compare the two treatments represented thereby would be to simply integrate the signals from each of the pixels and compare the integrated values. This would produce the essentially the same comparison that could be obtained without imaging the fluorescence radiation, i.e., if CCD camera 58 were replaced by a simple UV detector. In these particular examples, this would yield a ratio of treatments of 0.938, as the 20%-decrease in the bright zones is masked by the noise, by the slight increase in brightness of the dark zones and by the values for the zones in which there no change in fluorescence.

Another, more targeted, method would be to record the pixel values in each case and take a ratio of the maximum pixel values in each case. These maximum values will almost certainly occur in the pixels representing bright zones, where the 80% decrease, here, has been arbitrarily introduced. In the examples of FIG. 7 and FIG. 8, this would provide a ratio of about 0.897. This is certainly more indicative of the decrease than is provided by the averages but indicates about a 10% decrease compared with the known 20% decrease.

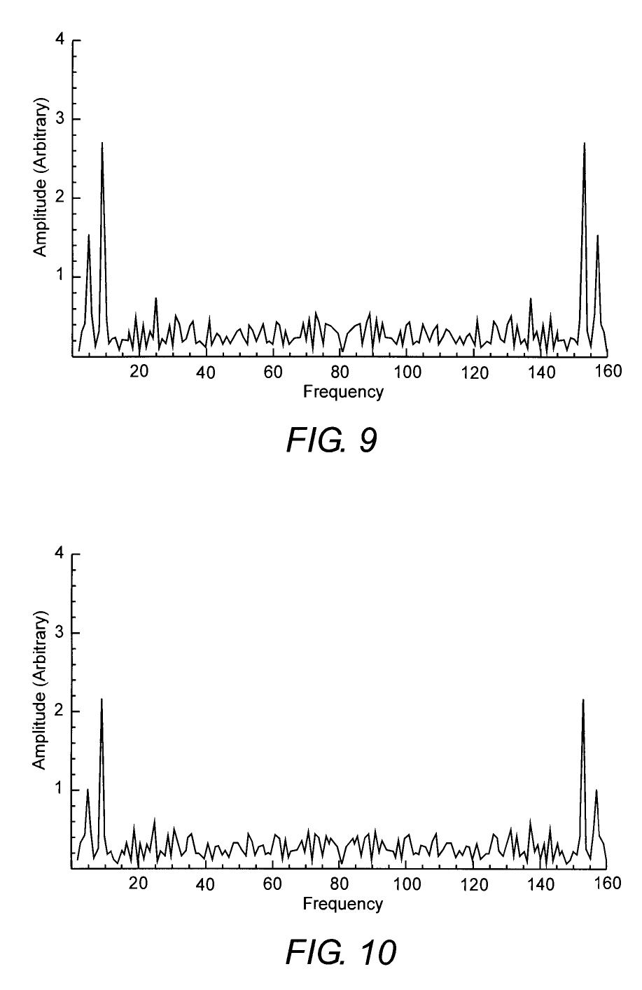

Another image processing method for detecting the fluorescence increase is to apply to the image data an algorithm, such as a Fourier transform, that can isolate from the untreated background the periodicity of distribution of the fluorescence introduced by fractional photo-therapy treatments. By way of example, FIG. 9 and FIG. 10 are graphs schematically representing Fourier transforms (frequency as a function of amplitude) formed from the data of FIG. 7 and FIG. 8 respectively. It can be seen that in each transform-graph there is a strong peak at frequency 9 (8+1) and a symmetrical peak at frequency 153 (160+1−8). The number 8, here, being the number of dark zones (periodic minima) in each line of the corresponding data arrays, with the number 160 being the number of data points (pixels) per line. Accordingly, it is to be expected that the amplitude of these peaks will be representative of the “real”, i.e., free-of-noise, amplitude in these bright zones. Indeed, in these particular examples, the ratio of the peak amplitudes at frequency 9 (and at frequency 153) is about 0.807, and provides a relatively accurate indication of the known ratio of 0.8.

Other examples of image processing methods include edge identification methods, contrast enhancement methods, two-dimensional Fourier transforms, and application of other mathematical filters such as those that are implemented in commercial photographic image processing and mathematical software. In other image analysis methods in accordance with the present invention, multi-wavelength illumination or filtering multiple wavelength ranges from stimulated fluorescence can be used to create two or more different spectral images that can be compared or mathematically processed using pixel-by-pixel subtraction or division of the spectral images. Such a multi-image approach can highlight the effects of different fluorophores and can allow the mathematical removal in processed data of baseline changes that are not due to treatment.

In considering any image processing methods of the present invention, it should be realized that it is possible that there will be some polarization sensitivity of the fluorescence radiation being imaged. This would be preferentially detected by arranging the illumination (fluorescence stimulating) radiation and the imaging of fluorescence to be non-collinear. This effect may be very subtle. Polarization-selectivity may possibly also be used to reduce “clutter” in a recorded image between skin-surface fluorescing features, for example lipids and serum, and fluorescing structures buried deeper in the epidermis and dermis. Scattering properties of the skin may also obscure any polarization-dependence of the fluorescence.

It is emphasized, here, that the above discussed example, wherein data is processed by Fourier transform is but one example of imaging processing that exploits the regular periodic distribution of the MTZs that is common in many fractional photo-therapy treatments. It should be noted, however, that fractional photo-therapy can also be effective if MTZs are not regularly spaced, in which case there may not be any periodicity content of a fluorescence image. There would, however, be some image characteristic representative of whatever was the spacing of the MTZs. In such a case other image processing algorithms, as noted above, or comparison of two different images may be used to highlight image characteristics due to the MTZ spacing.

By way of example, two images taken at different wavelengths may be electronically compared. The different wavelengths may be different fluorescence wavelengths of a single fluorophore or different wavelengths resulting from fluorescence of two different fluorophores. Two different images taken at different polarization states of the same fluorescence wavelength may also be compared. The image comparison may include adding, subtracting, dividing or multiplying the pixel values for the two images, or dividing the difference by the sum.

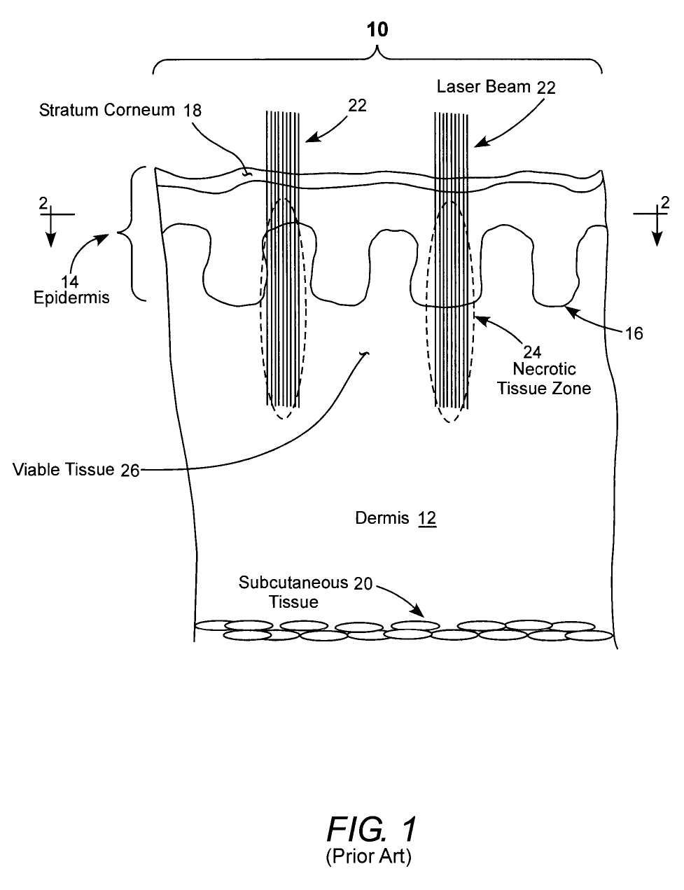

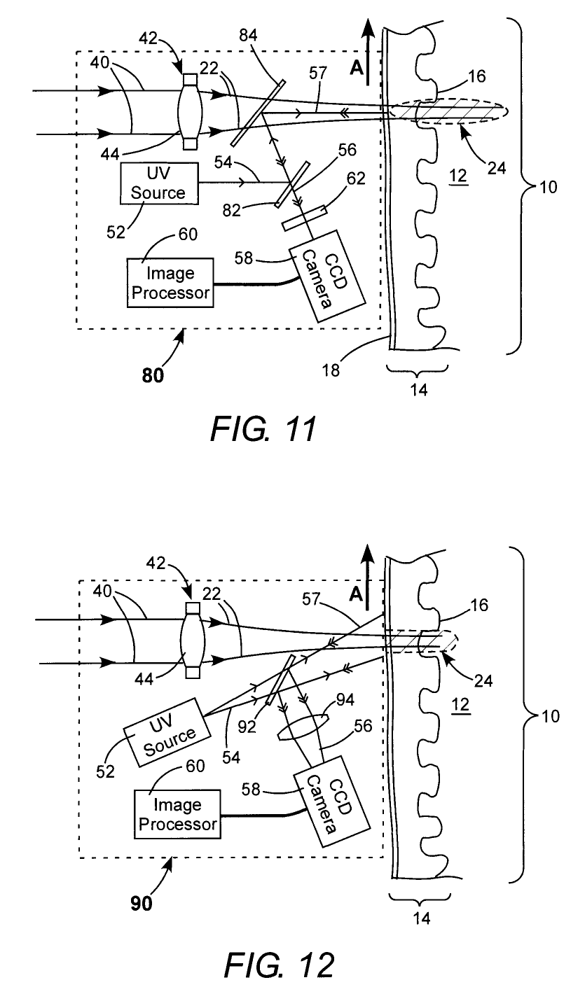

Returning now to a description of apparatus for implementing the monitoring method of the present invention, FIG. 11, schematically illustrates another preferred embodiment 80 of a fractional photo-therapy handpiece in accordance with the present invention. Handpiece 80 is similar to handpiece 50 of FIG. 1 with exceptions as follows. In handpiece 50 there are separate paths at skin 10 for treatment radiation, fluorescence-stimulating radiation, and fluorescence being imaged. In handpiece 80 there is a common path 57 for these radiations at skin 10. A dichroic beamsplitter 82 combines the paths (only an axial one thereof shown in FIG. 11) of fluorescence-stimulating radiation 54 and fluorescence 56 being imaged. Another dichroic beamsplitter 84 combines the combined paths of the fluorescence-stimulating radiation 54, and the fluorescence 56 being imaged with the path of a treatment beam 22. Dichroic beamsplitter 82 preferably is coated for maximum reflection of wavelengths between and 300 and 385 nm, and for maximum transmission at the wavelength of the fluorescence of the fluorophore being imaged, for example at 460 nm in the case of NAD-H. Dichroic beamsplitter 84 is preferably coated for maximum reflection of wavelengths between and 300 and 385 and the wavelengths of the fluorescence of the fluorophore being imaged, and for maximum transmission at the wavelength of the treatment radiation. The handpiece arrangement of FIG. 11 has an advantage that the treatment, fluorescence-stimulating, and imaging can be precisely co-registered for different working distances because they are collinear.

FIG. 12 schematically illustrates another preferred embodiment 90 of a fractional photo-therapy handpiece in accordance with the present invention. Handpiece 90 is similar to handpiece 80 of FIG. 11, with an exception that only the paths of fluorescence-stimulating radiation 54 and imaging radiation (fluorescence) 56 are combined at skin 10. Additionally, bandpass filter 62 of handpiece 80 is omitted in handpiece 90 and a dichroic mirror 92 is substituted for dichroic mirror 82 of handpiece 80. In a preferred arrangement for imaging fluorescence of NAD-H, dichroic mirror 92 has a maximum transmission for wavelengths between about 340 and 360 nm (a preferred fluorescence-stimulating wavelength range for NADH) and a maximum transmission at the peak wavelength of the stimulated fluorescence, i.e., at a wavelength of about 460 nm for NAD-H. Here, it should be noted that practical UV-multiplexing devices are typically more efficient when the shorter wavelengths are multiplexed in using reflection, rather than in transmission, and that the power of fluorescence-stimulating radiation is limited only by the power of available sources, whereas the fluorescence produced may be attenuated or masked by any above-discussed factors. In FIG. 12, MTZ 24 is depicted as extending from stratum corneum 18 through the epidermis. This can occur, for example, when treatment beam 22 is a low numerical aperture beam and no active skin cooling is employed. This form of the MTZ is not connected with the method of fluorescence stimulation and can occur in other embodiments of the inventive apparatus described herein.

In embodiments of the inventive apparatus described above with reference to FIGS. 4, 11, and 12, fluorescence-stimulating radiation can be delivered (and a fluorescence-image recorded) together with treatment radiation to an area of tissue being treated. It may be found advantageous, however, to deliver the fluorescence stimulating radiation before or after, or both before and after, the treatment radiation is delivered to the tissue. Delivering fluorescence-stimulating radiation (and recording a fluorescence-image) after the treatment radiation is delivered provides time for skin chemistry to react to the delivery of the treatment radiation and accordingly can provide a clearer indication of the structure of the associated fluorescence image related to the pattern of deposition of the treatment radiation. Delivering fluorescence-stimulating radiation before the treatment radiation is delivered provides a means of making a fluorescence image that can be used for comparison with a second fluorescence image made during or after delivery of treatment radiation.

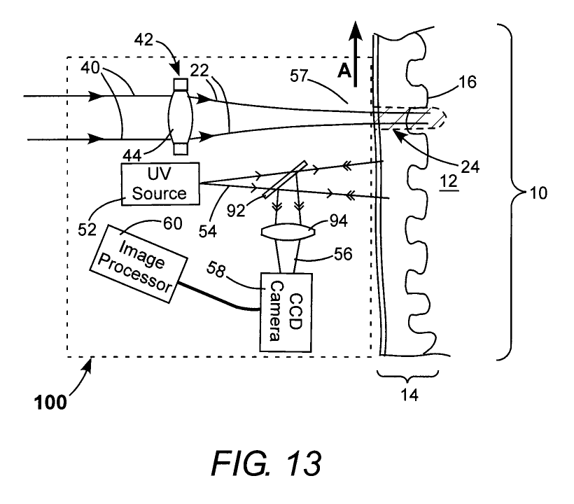

FIG. 13 shows still another embodiment 100 of apparatus in accordance with the present invention in which fluorescence-stimulating radiation is delivered to tissue, after delivery of treatment radiation. Apparatus 100 is similar to apparatus 90 of FIG. 12 with an exception that the orientation of the fluorescence-image generating components of the apparatus is arranged such that fluorescence-stimulating radiation can be delivered to tissue after delivery of treatment radiation. Those skilled in the art will recognize, without further illustration or detailed description, by adding a fluorescence-stimulating radiation source and a second CCD camera to apparatus 100, with fluorescence radiation being delivered ahead (in the direction of arrow A) of treatment radiation, fluorescence images could be recorded before and after delivery of treatment radiation.

Those skilled in the art to which the present invention pertains will recognize that the monitoring method of the present invention image may be used to identify regions of skin that have already been treated by fractional phototherapy, either during a prior treatment or a previous pass during the same treatment. Accordingly, those skilled in the art will also recognize that the inventive monitoring method may be used to control a fractional photo-therapy apparatus such that only regions that have not been previously treated are treated, for example, to maximize efficiency of use of the treatment energy. The inventive monitoring method may also be used to control fractional photo-therapy apparatus to provide precision dosage control, which in turn could be used to prevent over-treatment of a particular region of skin.

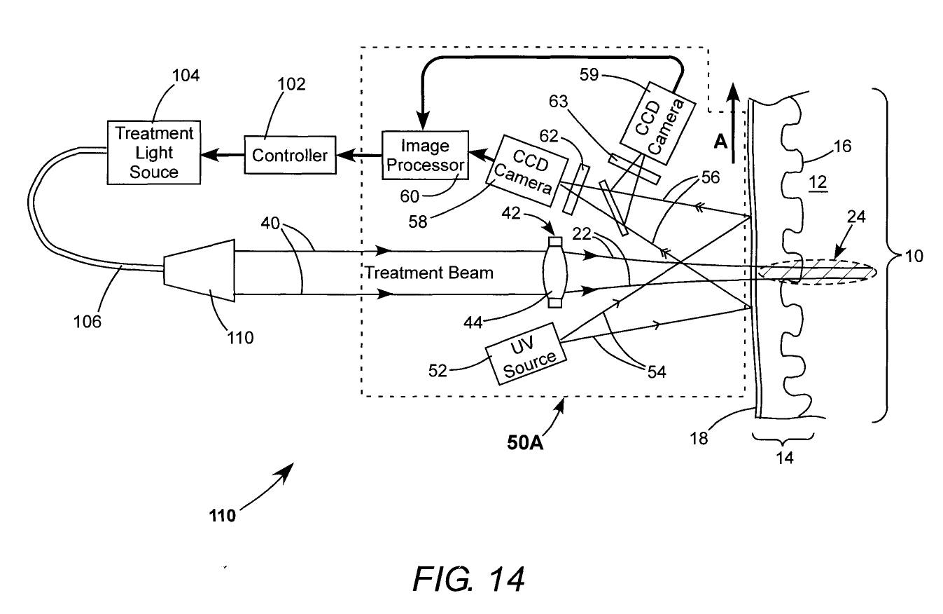

FIG. 14 schematically illustrates one preferred embodiment 100 of fractional photo-therapy apparatus controlled by fluorescence monitoring in accordance with the present invention. Apparatus 110 includes a handpiece 50A similar to handpiece 50 of FIG. 4 with an exception that handpiece 50A includes a second CCD camera 59 having a second bandpass filter 63 cooperative therewith for selecting a fluorescence wavelength range to be imaged. Image processor 60 can process images from both or any one of the CCD cameras for performing multi-spectral comparison as discussed above. The image processor is in communication with an electronic controller 102. Controller 102 in response to processed image information controls operation of treatment light (radiation) source 104. Radiation 40 from source 104, here, is delivered by an optical fiber 106 to a coupler 110. Coupler 110 delivers the radiation (treatment beam) to a microlens array 42 as described above with reference to FIG. 5 Control functions of controller include initiating or terminating delivery of radiation by the source varying parameters of the radiation in response to monitored progress of the phototherapy treatment.

Those skilled in the art to which the present invention pertains will recognize without further detailed description or illustration that handpiece 50A may be modified in certain ways to process one or more images, without departing from the spirit and scope of the present invention. By way of example, such modifications may include providing only a single CCD camera cooperative with a filter wheel including two or more bandpass filters having different passbands and recording, serially, two or more images at different wavelengths for processing. Alternatively the two CCD cameras may be retained and bandpass filters 62 and 63 replaced by polarizers arranged such that the CCD cameras record images in orthogonally opposed polarizations. Wavelength selective polarizers may be used to provide both spectral and polarization difference in two recorded images. It should also be noted that the image processing function of image processor 60 may be included in controller 102. Further, while in FIG. 14 the treatment light source is depicted as being separate from the handpiece, those skilled in the art will recognize that a treatment light source, such as a diode laser or a diode laser array, of sufficiently small dimensions may be incorporated in the handpiece. Those skilled in the art will recognize that reference to Fourier Transform throughout this document refers to discrete forms of the Fourier Transform because there are a discrete number of pixels.

In summary, the present invention is described above with reference to a preferred and other embodiments. Persons of ordinary skill in the art may modify the above-described embodiments without undue experimentation or without departing from the spirit or scope of the present invention. All such departures or deviations should be construed to be within the scope of the following claims.

What is claimed is:

1. A method of monitoring fractional photo-therapy treatment, the fractional phototherapy treatment including delivery of treatment radiation in a pattern of treatment zones to an area of tissue, the method comprising the steps of:
irradiating the area of tissue being treated with electro-magnetic radiation thereby stimulating emission of fluorescence radiation from one or more fluorophores in the area of tissue being treated;
recording one or more images of the area of tissue using the fluorescence radiation emitted from at least one of said fluorophores, said images having a spatial distribution of fluorescence depending on structural features of the area of tissue combined with features characteristic of the pattern of treatment-radiation delivery; and
electronically processing said one or more images to identify the fluorescence radiation in said one or more images resulting from the delivery of the treatment radiation.

2. The method of claim 1, wherein said irradiating and recording steps are carried out after delivery of the treatment radiation.

3. The method of claim 1, wherein treatment radiation zones in the pattern of treatment radiation are substantially equally spaced and said electronic processing includes analyzing at least one region of at least one of said one or more images to isolate periodically occurring features thereof resulting from the substantially equally spaced treatment radiation zones in the pattern of the treatment radiation.

4. The method of claim 3, wherein said analyzing includes generating a Fourier transform of said image region.

5. The method of claim 4, wherein said electronic processing includes interpreting a peak value of said Fourier transform as a measure of the progress of the fractional photo-therapy treatment.

6. The method of claim 4, wherein said electronic processing includes interpreting a peak value of said Fourier transform as a measure of the treatment radiation dose delivered to the area of tissue being treated.

7. The method of claim 1, wherein said fluorophores include at least one of tryptophan, porphyrins, NAD-H, flavins, elastin, and collagen and a majority of said fluorescent emission used for recording said images is emitted from said fluorophore.

8. The method of claim 1, wherein first and second images are recorded, said first image being recorded using fluorescence radiation in a first band of wavelengths characteristic of emission from a first of said fluorophores and said second image being recorded using fluorescence radiation in a second band of wavelengths characteristic of emission from a second and different of said fluorophores.

9. The method of claim 8, wherein said first fluorophore is reduced nicotinamide adenine dinucleotide (NAD-H), and said second fluorophore is elastin.

10. The method of claim 8, wherein said electronic processing includes one of adding, subtracting, dividing, and multiplying said first and second images.

11. The method of claim 8, wherein said first and second images are recorded using respectively first and second recording devices.

12. The method of claim 8, wherein said first and second images are recorded sequentially using a single recording device.

13. A method of monitoring fractional photo-therapy treatment, the fractional phototherapy treatment including delivery of treatment radiation in a regular pattern of spaced-apart zones to an area of tissue, the method comprising the steps of:
irradiating the area of tissue with electro-magnetic radiation thereby stimulating emission of fluorescence radiation from reduced nicotinamide adenine dinucleotide (NAD-H) in the area of tissue, the amount of NAD-H in the tissue in and around zones thereof to which treatment radiation is delivered being dependent on the amount of radiation delivered to the zones;
recording a first image of the area of tissue in a wavelength range characteristic of NAD-H fluorescence radiation, said first image having a spatial distribution of fluorescence including background features dependent on structural features of the area of tissue combined with regularly distributed features characteristic of the pattern of treatment-radiation delivery; and
electronically processing said first image to separate said regularly distributed features of said image from said background features and thereby provide a measure of the fluorescence in said image resulting from delivery of the treatment radiation to the area of tissue.

14. The method of claim 13, wherein said separation of said regularly distributed features from said background features includes generating a Fourier transform of a region of said image.

15. The method of claim 14, wherein said electronic processing includes interpreting a peak value of said Fourier transform as a measure of the progress of the fractional photo-therapy treatment.

16. The method of claim 14, wherein said electronic processing includes interpreting a peak value of said Fourier transform as a measure of the treatment radiation dose delivered to the area of tissue.

17. The method of claim 13, wherein said separation of said regularly distributed features from said background features includes the steps of recording a second image in a wavelength range characteristic of the fluorescence spectrum of elastin, the fluorescence of which is not substantially affected by delivery of treatment radiation thereto, such that said second image does not include features representative of said the pattern of treatment-radiation delivery; and comparing said second image with said first image.

18. Apparatus for photo-thermal treatment of tissue, comprising:
a source of treatment radiation;
an arrangement for delivering treatment radiation from said treatment radiation source to an area of tissue being treated;
a source of fluorescence-stimulating radiation, said fluorescence-stimulating radiation having a range of wavelengths selected to stimulate fluorescence from one or more fluorophores in the area of tissue being treated;
a camera for recording one or more images of the area of tissue being treated at a wavelength of the stimulated fluorescence;
electronic circuitry, cooperative with said treatment-radiation source and said camera; and
wherein said electronic circuitry is arranged to analyze said one or more recorded images and determine from said analysis the progress of the photo-thermal treatment in said area of skin being treated, and arranged to control said treatment-radiation source responsive to said determination.

19. The apparatus of claim 18, wherein said controlling of said treatment-radiation source includes preventing said treatment-radiation source from delivering radiation if said determination is that said area of tissue has already been treated.

20. The apparatus of claim 18, wherein such controlling of said treatment-radiation source includes altering parameters of the treatment radiation delivered by said treatment-radiation source in response to said determination.

21. The apparatus of claim 18, wherein said fluorescence-stimulating-radiation source and said camera are contained in delivery apparatus remote from said treatment-radiation source, wherein an optical arrangement is provided for transporting said treatment-radiation from said treatment-radiation source to said delivery apparatus, and wherein said delivery apparatus is arranged to deliver said treatment and fluorescence-stimulating radiations to tissue to be treated.
